(19) United States
(12) Patent Application Publication
(10) Pub. No.: US 2008/0240349 A1
(43) Pub. Date: Oct. 2, 2008

(54) Radiation Therapy System and Methods For Planning a Radiation Therapy of a Patient, and For Patient Positioning

(76) Inventors: Klaus Herrmann, Nurnberg (DE); Eike Rietzel, Darmstadt (DE); Andres Sommer, Langensendelbach (DE)

Correspondence Address: BRINKS HOFER GILSON & LIONE, P.O. BOX 10395, CHICAGO, IL, 60610, US

(21) Appl. No.: 11/995,809

(22) PCT Filed: Jul. 26, 2006
(86) PCT No.: PCT/EP2006/064664

§ 371 Date: May 15, 2008

Related U.S. Application Data
(60) Provisional application No. 60/702,379, filed on Jul. 26, 2005.

(30) Foreign Application Priority Data
Jul. 26, 2005 (DE) 10 2005 034913.7

(51) Int. Cl. A61N 5/10

(52) U.S. Cl. 378/65

(57) ABSTRACT

The present embodiments relate to a device for obtaining image data for planning a radiation therapy of a patient. The device includes a CT gantry and a patient positioning unit. The patient positioning unit is designed in such a way that the patient can be brought in space into any desired body position in which the radiation therapy is also carried out. The CT gantry is arranged in a freely moveable fashion in such a way that imaging for the purpose of radiation therapy planning can be carried out in this body position of the patient.

The present patent document claims the benefit of PCT Application No. PCT/EP2006/064664, filed Jul. 26, 2006, German Patent Application Serial Number DE 10 2005 034 913.7, filed Jul. 26, 2005, and U.S. Provisional Application Ser. No. 60/702,379, filed Jul. 26, 2005, all of which are hereby incorporated by reference.

### BACKGROUND

The present embodiments relate to a radiation therapy system having a device for obtaining image data for planning the radiation therapy, having an irradiation station having a therapeutic beam delivery device and having a patient positioning unit. The present embodiments relate, furthermore, to methods for planning a radiation therapy of a patient, and for patient positioning.

Computer tomography units (CT units) are used in radiation therapy planning for the computer-aided generation of 3D image data of a patients body area that contains a tumor. The image data are used to establish the irradiation fields required for a successful irradiation of the tumor, i.e. the intensity and direction of incidence of the radiation. Use is made in this case of the fact that the attenuation of the imaging X-rays can be scaled to the interaction of the therapeutic beam with the transirradiated tissue.

It is preferred to use a CT for therapy planning since; for a large volume, it provides a high resolution measurement of the attenuation of the radiation by X-ray detectors as the body is penetrated; the radiation is emitted by an X-ray source from various directions onto the patient. The X-ray source and the X-ray detectors rotate about the patient lying in a measurement opening, and so measurements that lead to the high resolution 3D image data record are carried out from a multiplicity of directions.

With state of the art CT, the patient is typically located during examination in a lying posture on a patient couch positioned in the measurement opening. In order for the anatomy to correspond during the irradiation to the anatomy used for the planning, the irradiation is also performed in this position. For irradiation purposes, use is made of irradiation stations with a fixed irradiation direction and, of gantry-based irradiation stations in which irradiation is possible from various directions by rotating the beam of the gantry about the patient. In conventional radiation therapy, irradiation is performed with X-ray, and in the case of particle therapy use is made, for example, of protons, neutrons and carbon ions.

Depending on the irradiation station planning the directions of incidence for the therapeutic beam is restricted and, because of the underlying planning imaging, the positioning of the patient is also restricted. A change in the patient's position usually entails an internal displacement of internal organs. It is therefore to be recommended to carry out the planning imaging in the irradiation position in order to acquire the anatomy in the irradiation position as accurately as possible.

DE 101 46 915 A1 has already disclosed an X-ray apparatus, in particular a computer tomography apparatus, that is provided for examining a patient in an erect or partially erect posture and thus to the accompaniment of a natural, static loading of a specific body area. It is possible in this case to tilt the plane of rotation of the apparatus in order to align the measurement planes. DE 101 46 915 A1 discloses an imaging unit (for example C-arc X-ray apparatusor CT apparatus) that permits recordings in an erect or partially erect posture.

EP 0 220 501 B1 discloses an X-ray diagnostic system with an adjustable X-ray tube, an adjustable image recording system and a patient couch (support). These system components may be moved individually in three dimensions with the aid of actuating device that can be operated by a motor.

WO 99/53997 discloses a method for irradiating a patient with the aid of a horizontal particle beam by using a fixing unit to align the patient in relation to the beam in such a way that the body axis is perpendicular to the particle beam. A similar device is disclosed in US 2005/018735 A2, and in this case a robot arm with six degrees of freedom is used to position the patient.

### SUMMARY AND DESCRIPTION

The present embodiments may obviate one or more drawbacks or limitations of the related art. For example, in one embodiment, the restrictions with regard to the direction of incidence in the case of radiation therapy are neutralized, in particular for irradiation stations with a fixed beam direction.

In one embodiment, a radiation therapy system for carrying out a radiation therapy of a patient includes a device for obtaining image data for planning the radiation therapy of a patient in a posture adopted by the patient, which can be aligned freely in space into a body position with the aid of a patient positioning unit of the device. The device also includes an imaging device that can be aligned in a freely moveable fashion such that imaging can be carried out in the posture of the patient that is aligned in space. The radiation therapy system includes a storing unit for storing at least one parameter of the patient positioning unit during planning. The parameter determines the alignment of the posture in space. The radiation therapy system also includes an irradiation station having a therapeutic beam delivery device in front of which the patient can be brought in space into the same alignment of the posture on which the planning was based with the aid of the same patient positioning unit or a further patient positioning unit. The patient positioning unit reads out the at least one parameter from the storing unit and undertakes an alignment corresponding to the parameter.

In one embodiment, the device for obtaining image data may be used with a CT gantry to examine a patient in any desired position in order to obtain image data for planning a radiation therapy. Since the radiation therapy system stores at least one “setting” parameter, which determines the alignment of the posture in space, of the patient positioning unit for planning imaging, it is possible to position the patient in virtually the same posture and alignment for the irradiation.

Instead of implementing the direction of incidence of the therapeutic beam in relation to the main patient axis by rotating the therapeutic beam about the patient, the patient may be aligned as desired, and thus, in particular, for the direction of incidence to be tuned to the anatomy even at irradiation stations with a fixed beam direction, and to be freely selected. Restrictions then still occur essentially only with regard to alignments that are unreasonable for the patient.

In the alignment for the irradiation, the patient is also covered for imaging during therapy planning, since otherwise the internal organs could shift in position between planning imaging and irradiation on the basis of a change in position of the patient. The tilt angles of the patient or the patient couch (support) that form the basis of the irradiation planning are part of the irradiation data that characterize an irradiation operation. The patient is preferably irradiated in the alignment on which the planning was based if no relatively small correction in position takes place.

Since it is possible to dispense with gantry irradiation stations, the design of a radiation therapy system is simplified and yet comparable in flexibility in the case of irradiation. In the case of particle therapy systems this design leads to large savings in cost, to a flexibility in the selection of the direction of the incident beam, and thus to a good separation between tissue that is irradiated and that which is not to be irradiated.

The present embodiments may dispense with the gantry and improve the irradiation because the irradiation planning is free of restrictions in the selection of the posture in which the irradiation is subsequently carried out.

In one embodiment, a patient is X-rayed in space in any desired body position in order to obtain image data. The body position describes the alignment of the patient in space. For example, the body position may comprise the posture of the patient, for example on the patient couch, and, the three-dimensional position of the patient couch (with the patient) in space. Starting from this body position, a CT gantry with a radiation source and radiation detector is moved along the main axis of the patient relative to the patient (or the patient is moved relative to the gantry) in order to obtain image data. Depending on the embodiment of the device for obtaining image data for planning a radiation therapy, the CT gantry may be brought over the patient couch before then assuming the appropriate body position by tilting both the CT gantry and the patient couch. Alternatively, the imaging can be performed, for example, with the aid of a cone beam CT unit. The therapy planning is performed on the basis of these image data, for example, the fixing of the direction of incidence of the therapeutic beam, and of the radiation intensity, in the case of particles, for example, the energy of the particles. Possible directions of incidence are prescribed by the irradiation device provided for use, for example, a particle beam in a horizontal direction or at 45° from above, or a gantry having angles that can be set in a plane of rotation.

### BRIEF DESCRIPTION OF THE DRAWINGS

Further advantageous, features and details of the present embodiments will become evident from the description of illustrated exemplary embodiments given herein and the accompanying drawings, which are given by way of illustration only and thus are not limitative of the present embodiments, wherein:

FIG. 1 illustrates one embodiment of a radiation therapy system having a device for obtaining image data, and

FIG. 2 is a flowchart that illustrates the operation of such a radiation therapy system.

### DETAILED DESCRIPTION

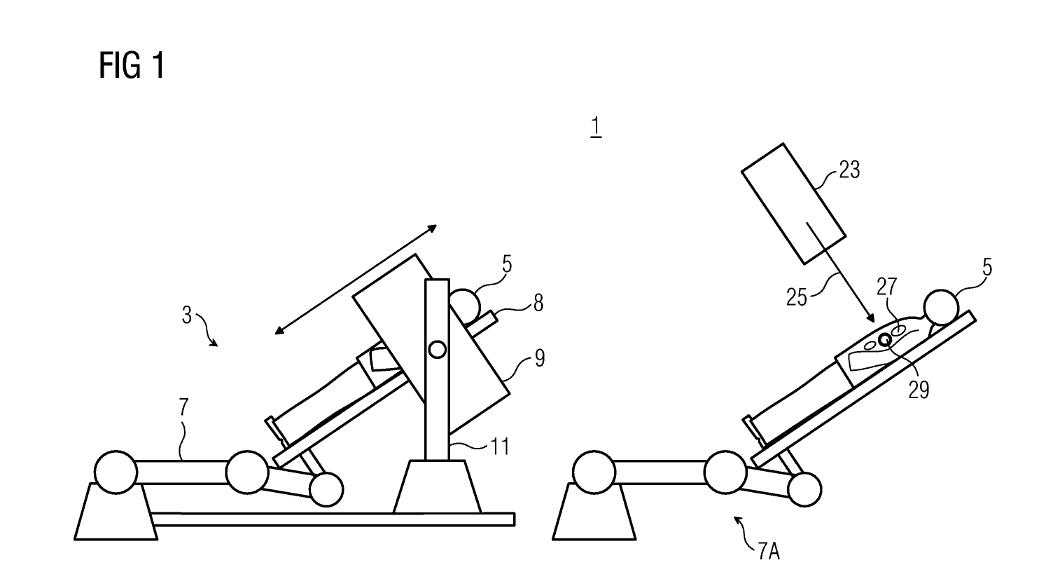

FIG. 1 shows a radiation therapy system 1 having an imaging system 3 for obtaining image data for planning a radiation therapy of a patient 5. The imaging system 3 comprises a patient positioning unit 7 that is designed in such a way that the patient 5 can be brought in space into any desired body position. A CT gantry (imaging device) 9 (or a cone beam CT unit) is used in this body position in order to obtain image data that is used to plan a radiation therapy of the patient 5. The CT gantry 9 is freely moveable in such a way that it is possible to carry out imaging for the purpose of radiation therapy planning in the body position of the patient 5. For example, the CT gantry 9 is held via a frame 11 in a fashion capable of rotation, displacement and tilting. A relative movement of the CT gantry or its X-ray emitters and X-radiation detectors with reference to the patient 5 can be performed via the patient positioning unit 7 through a displacement of the patient, or by a movement of the CT gantry 9.

The radiation therapy system 1 further has an irradiation station at which the irradiation is carried out. The positioning can be carried out with the aid of the same or of a further patient positioning unit (7A in FIG. 1). The flexibility with regard to the alignment of the patient in his posture during the planning imaging also permits the irradiation to be carried out in a correspondingly free patient position. The patient positioning unit 7 may be a robot that permits a free alignment and movement of the patient 5, the patient being supported, in particular, in a fashion lying on a patient couch 8 of the patient positioning unit 7 or in a fashion sitting in a patient seat of the patient positioning unit 7, or standing.

The radiation therapy system 1 may include a radiation therapy planning unit for carrying out radiation therapy planning 19 on the basis of the image data record. An appropriate computer-aided planning unit in the vicinity of the planning imaging and/or the irradiation station permits the radiation therapy to run in a simple way.

The radiation therapy system may include a storage unit that stores at least one setting parameter, determining the alignment of the posture in space, of the patient positioning unit 7 of the planning imaging. This allows the patient to be positioned in virtually the same posture and alignment for the irradiation. The patient positioning unit 7, 7A reads out at least one setting parameter from the means for storage and under-takes an alignment corresponding to the setting parameter. The storage unit may be connected to a control system of the radiation therapy system via a data transmission system, or form a part of the control system, which is designed for driving, inter alia, patient positioning device(s), radiation source(s) and beam exit(s).

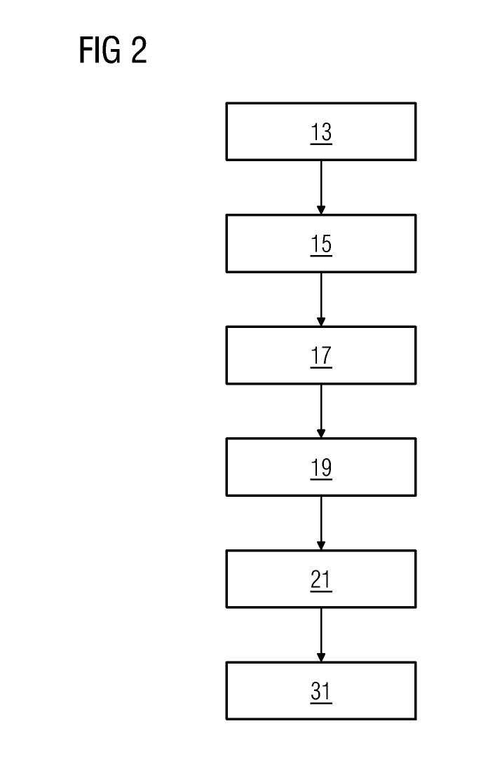

The method illustrated in FIG. 2 may be completed during operation of the radiation therapy system 1. After the prompting 13 of any desired body position by the patient 5, an imaging device (for example the CT gantry 9 in FIG. 1) is aligned 15 with the body position of the patient 5. The body position is to be understood both as the posture of the patient 5 on, for example, a patient couch 8 or a patient seat, and the alignment of such a patient posture in space.

A movement 17 of the patient 5 relative to the imaging device 9 is used to obtain image data that, for example, permit the generation of 3D images of the patient that are suitable for therapy planning. With the aid of the image data, an irradiation plan 19 is compiled that fixes the direction of incidence of the therapeutic radiation and further beam parameters—in a fashion designed for the body position present during the planning imaging. Subsequently, the patient 5 is positioned 21 in the irradiation position near an exit 23 of a therapeutic beam 25. Use is made of, for example, an identical patient positioning unit 7A on which the patient 5 is positioned either in the same body position on which the therapeutic planning was based, or in an irradiation position that comes very near this body position. In this irradiation position, which preferably corresponds to the body position, for example, organs 27 of the patient 5 are now arranged spatially in the body in relation to a tumor 29 to be irradiated in such a way that the irradiation stresses these organs 27, or body tissue to be spared, as little as possible.

A radiation therapy 31 is finally carried out in the irradiation position with the aid of the therapeutic beam 25.

Possible therapeutic beams 25 are particle beams, for example, protons or carbon ions or high energy X-radiation. The beam exit 23 can be, for example, a fixed-beam beam exit. A fixed beam exit fixes the direction of the therapeutic beam. Alternatively, the beam exit 23 can be the beam exit of a gantry with the aid of which the beam exit can be rotated about the patient in a plane.

Various embodiments described herein can be used alone or in combination with one another. The forgoing detailed description has described only a few of the many possible implementations of the present invention. For this reason, this detailed description is intended by way of illustration, and not by way of limitation. It is only the following claims, including all equivalents that are intended to define the scope of this invention.

1. A radiation therapy system for carrying out a radiation therapy of a patient, having
an imaging system for obtaining image data for planning the radiation therapy of a patient a posture adopted by the patient, the imaging system including a patient positioning unit that is operative to align the patient freely in space into a body position, and an imaging device that can be aligned in a freely moveable fashion such that imaging may be carried out in the posture of the patient that is aligned in spaces;
a storing unit that is operative to store least one parameter of the patient positioning unit during planning, said parameter determining the alignment of the posture in space; and
an irradiation station having a therapeutic beam delivery device, the same patient positioning unit or a further patient positioning unit operable to bring the patient in front of the therapeutic beam delivery device in space into the same alignment of the posture on which the planning was based, the same or further patient positioning unit being operative to position the patient based on the at least one parameter from the storing unit and to align the patient corresponding to the at least one parameter.

2. The radiation therapy system as claimed in claim 1, comprising a radiation therapy planning unit operative to plan the radiation therapy on the basis of the image data record.

3. The radiation therapy system as claimed in claim 1, characterized in that the patient positioning unit comprises a robot that permits a free alignment and movement of the patient, who is lying on a patient couch of the patient positioning unit for sitting in a patient seat of the patient positioning unit, or standing.

4. The radiation therapy system as claimed in claim 1, wherein the imaging device includes a CT gantry or a cone beam CT unit, the imaging device may be displaced horizontally and/or vertically, and/or along an adjustable direction, and/or may be tilted about an adjustable axis such that image planes of the imaging are arranged perpendicular to the arbitrarily adjustable patient alignment.

5. A method for planning a radiation therapy of a patient the method comprising:
positioning the patient in any desired body position that is substantially the same as a irradiation position or corresponds to the irradiation position,
aligning an imaging device with the body position,
moving the patient relative to the imaging device in conjunction with image data of the patient being simultaneously obtained by the imaging device, and
planning the radiation therapy using the image data for the irradiation position.

6. The method as claimed in claim 5, wherein positioning the patient in the body position includes assuming a posture of the patient on a patient holder or on a patient seat, and aligning the patient and the patient holder in the posture.

7. The method as claimed in claim 6, wherein said alignment of the patient in the posture is undertaken via a robot unit that acts on the patient holder.

8. The method as claimed in claim 7, wherein the body position enables the patient's body to receive a therapeutic beam path of a therapeutic beam of a radiation therapy system.

9. The method as claimed in claim 8, wherein during alignment of the imaging device, a CT gantry of the imaging device is positioned in such a way that the patient is arranged on the axis of symmetry of the CT gantry.

10. The method as claimed in claim 9, wherein the patient and/or imaging device are/is moved during the relative movement.

11. A method for patient positioning during a radiation therapy of a patients, the method:
positioning the patient in any body position that is substantially the same as an irradiation position or corresponds to the irradiation position,
aligning an imaging device with the body position,
moving the patient relative to the imaging device while simultaneously obtaining image data by the imaging device,
compiling an irradiation plan on the basis of the image data obtained for the irradiation position that is substantially the same as the body position or corresponds to the body position, and
positioning the patient in the irradiation position.

12. The method as claimed in claim 6, wherein the patient holder includes a patient support.

13. The method as claimed in claim 8, wherein the therapeutic beam of a radiation therapy system includes a fixed, particle beam.
